(19) United States
(12) Patent Application Publication
(10) Pub. No.: US 2025/0188047 A1
(43) Pub. Date: Jun. 12, 2025

(54) METHOD FOR PREPARING 5-ACETOXYMETHYL FURFURAL (AMF) FROM SUGAR INGREDIENTS AND METHOD FOR PREPARING 2,5-FURANDICARBOXYLIC ACID (FDCA) COMPRISING SAME

(71) Applicant: KOREA RESEARCH INSTITUTE OF CHEMICAL TECHNOLOGY, Daejeon (KR)

(72) Inventors: Dong Won HWANG, Daejeon (KR); In Yong EOM, Daejeon (KR); Ji Hoon KIM, Daejeon (KR); Ma Eum LEE, Daejeon (KR); Young Kyu HWANG, Daejeon (KR); Yeo Jin KIM, Daejeon (KR)

(73) Assignee: KOREA RESEARCH INSTITUTE OF CHEMICAL TECHNOLOGY, Daejeon (KR)

(21) Appl. No.: 18/844,767

(22) PCT Filed: Mar. 7, 2023
(86) PCT No.: PCT/KR2023/003112

§ 371 Date: Sep. 6, 2024

(30) Foreign Application Priority Data
Mar. 11, 2022 (KR) 10-2022-0030648

(51) Int. Cl. C07D 307/48; C07D 307/68

(52) U.S. Cl. CPC C07D 307/48; C07D 307/68

(57) ABSTRACT

The present invention relates to a method for preparing 5-acetoxymethyl furfural (AMF) from sugar ingredients and, specifically, to a method for preparing 5-acetoxymethyl furfural (AMF) from sugar ingredients by converting XMF into AMF without separation and purification, which remove an extraction solvent from a solution in which an aqueous phase comprising 5-halomethyl furfural (XMF) and an organic phase are mixed, thereby enabling XMF in a crude product form to be directly used without requiring in-process separation and purification equipment.

### TECHNICAL FIELD

The present disclosure relates to a method of preparing 5-acetoxymethyl furfural (AMF) from a sugar ingredient, in which crude 5-halomethyl furfural is converted into AMF without performing separation and purification to remove an extraction solvent from an organic phase containing 5-halomethyl furfural, thereby enabling 5-halomethyl furfural in a crude or crude product form to be directly used without requiring in-process separation and purification equipment.

### BACKGROUND ART

With global warming, occurring around the world, being pointed out as the probable cause of rapidly changing ecosystems and living environments along with abnormal climate, research and development continue to be in progress on discovering and utilizing renewable and sustainable resources instead of fossil fuel-derived resources, which are the cause of global warming.

The abundant plant and woody biomass on Earth contains large amounts of carbohydrates. These carbohydrates, which are polysaccharides, include monosaccharides such as glucose, galactose, and fructose. Such monosaccharides contain highly reactive hydroxyl groups (—OH) and thus are convertible through various chemical reactions, leading to attempts to use monosaccharides as useful chemical raw materials in industry and daily life or to prepare high-value-added compounds.

5-Hydroxymethylfurfural (hereinafter referred to as HMF), prepared from dehydration reactions of the sugar ingredient of monosaccharides, is widely known as a platform compound that is convertible into furan-based compounds. HMF is convertible into various derivatives such as 2,5-furandicarboxylic acid (FDCA), 2,5-furandimethanol (FDM), and 5-ethoxymethylfurfural (EMF).

However, there have been problems in that HMF converted from sugars are readily converted into levulinic acid and formic acid through dehydration reactions or forms polymers (humins) through aldol condensation reactions. There is a technology known to solve this problem, which is to react the HMF with a halogen compound for conversion into 5-halomethyl furfural (hereinafter referred to as XMF) and then use a two-phase solution system for extraction involving an organic solvent to ultimately obtain an XMF form.

In a non-patent document (ChemSusChem, 2015, 8, 1179-1188) regarding From Lignocellulosic Biomass to Furans via 5-Acetoxymethylfurfural as an Alternative to 5-Hydroxymethylfurfural by E S Kang et al., AMF was obtained by introducing biomass into a mixed solution of HCl and trichloroethane (TCE) to prepare chloromethylfurfural (CMF), extracting CMF from the separated organic layer, separating and purifying the extracted CMF, and then reacting the resulting CMF with alkyl ammonium acetate. However, the non-patent document described above has a problem in that the step of separating and purifying CMF must be involved.

In order for technology for preparing AMF from XMF to be applied to biomass-derived preparation processes by overcoming the problems described, there is a need to conduct research and development on technology that enables the economic feasibility of the AMF preparation process to be obtained while preparing AMF in high yields.

### DISCLOSURE

### Technical Problem

A method of preparing AMF of the present disclosure aims to provide an AMF preparation process capable of preparing AMF in high yields without performing separation and purification processes to remove an extraction solvent.

### Technical Solution

To solve the problems described above, the present disclosure may be characterized in that a method of preparing AMF from a sugar ingredient includes the following steps: (a) in a two-phase solvent system including an aqueous phase and an organic phase of an organic extraction solvent having a relative polarity of 0.2 or less based on a polarity of water at 1, converting a sugar ingredient to prepare XMF from the aqueous phase and then extracting the prepared XMF into the organic phase; and (b) adding acetate ions (AcO⁻) to the organic phase extracted directly in Step (a) to convert the extracted XMF into AMF.

In the embodiment of the present disclosure, the extraction solvent having a relative polarity of 0.2 or less in Step (a) may include one or more from toluene, xylene, and chlorobenzene.

In the embodiment of the present disclosure, the XMF may be characterized by being bromomethylfurfural (BMF).

In the embodiment of the present disclosure, the acetate ions (AcO) in Step (b) may be characterized by being present as exchange groups in an anion exchange resin.

In the embodiment of the present disclosure, an XMF/AcO⁻ molar ratio in Step (b) may be in the range of 0.01 to 0.99.

In the embodiment of the present disclosure, the anion exchange resin may be characterized by being substituted through solvent substitution. In this case, the solvent substitution may involve a step of substituting the anion exchange resin through solvent substitution using one or more solvents selected from a ketone, an alcohol, and an ether that are miscible solvents in water and the extraction solvent.

In the embodiment of the present disclosure, the conversion of the extracted XMF into AMF may be performed under a condition in a temperature range of room temperature (25° C.) to 110° C.

In the meantime, the present disclosure provides a method of preparing FDCA from a sugar ingredient, the method including the following steps: (A) in a two-phase solvent system including an aqueous phase and an organic phase of an organic extraction solvent having a relative polarity of 0.2 or less based on a polarity of water at 1, converting a sugar ingredient to prepare XMF from the aqueous phase and then extracting the prepared XMF into the organic phase; (B) adding acetate ions (AcO⁻) to the organic phase extracted directly in Step (A) to convert the extracted XMF into AMF; (C) recovering the resulting AMF converted from the organic phase; and (D) converting the recovered AMF into FDCA through oxidation.

In the embodiment of the present disclosure, the oxidation in Step (D) may be performed under a condition in a temperature range of 70° C. to 130° C.

In the embodiment of the present disclosure, the oxidation in Step (D) may be performed in the presence of a catalyst under a condition at 1 to 20 bar of oxygen or at 1 to 50 bar of air.

### Advantageous Effects

According to the present disclosure, AMF can be prepared by bringing acetate ions into contact with XMF in an XMF-containing extraction solvent without performing separation and purification to separate and remove the extraction solvent, thus simplifying the AMF preparation process by not requiring separation and purification equipment and enabling cost reduction.

In addition, after preparing AMF using XMF in a crude product form, FDCA can be prepared through an additional oxidation reaction.

Furthermore, when an acetate anion (AcO−)-exchange resin substituted through solvent substitution is used as a reactant when preparing AMF, halide reactive groups of XMF can be selectively substituted with acetate anions of the moisture-free ion exchange resin. For this reason, side reactions occurring due to moisture are kept to a minimum, leading to an AMF yield improvement effect. Moreover, the yield improvement effect described above can be multiplied by selecting a halogen species of XMF or creating an appropriate XMF/AcO molar ratio condition.

### DESCRIPTION OF DRAWINGS

FIG. 1 is a diagram showing a reaction process in which AMF is prepared from a sugar ingredient;

FIG. 2 shows CMF conversion rate, AMF selectivity, and AMF yield in Examples 1 and 2 and Comparative Examples 1 and 2 as one example of the present disclosure, as a graph;

FIG. 3 shows the effects of molar ratios of CMF/AcO⁻ in an exchange resin on AMF yield and conversion speed depending on reaction time as one example of the present disclosure, as a graph;

FIG. 4 shows the effects of molar ratios of BMF/AcO⁻ in an exchange resin on AMF yield and conversion speed depending on reaction time as another example of the present disclosure, as a graph; and

FIG. 5 shows AMF conversion rate, FDCA yield, and 5-formyl-2-furancarboxylic acid (FFCA) yield as a result of an oxidation reaction performed on an AMF-containing reaction product solution in Example 1 as one example of the present invention, as a graph.

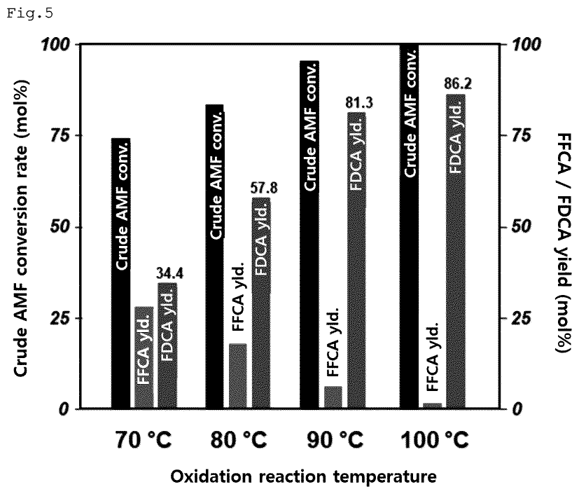

### BEST MODE

Unless defined otherwise, all terms including technical and scientific terms used herein have the same meaning as commonly understood by those skilled in the art to which the present disclosure belongs. In general, the nomenclature used herein is well-known and commonly used in the art.

Unless the context clearly indicates otherwise, it will be further understood that the terms “comprises”, “comprising”, “includes”, and “including”, when used herein, specify the presence of components, but do not preclude the presence or addition of one or more other components.

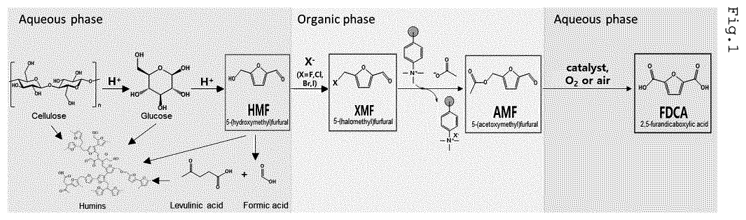

FIG. 1 is a diagram showing a reaction process in which AMF is prepared from a sugar ingredient.

According to FIG. 1, D-glucose, one of the sugar ingredients, is converted into HMF through a continuous dehydration reaction. In this case, as the dehydration reaction further proceeds, HMF is converted into levulinic acid. However, HMF form has a wider range of industrial uses than levulinic acid, so HMF is in greater demand. Therefore, as a method to prevent the conversion of HMF into levulinic acid while selectively obtaining HMF, there has been a known technology to indirectly obtain HMF by substituting a hydroxyl group (—OH) in HMF with a halogen atom (F, Cl, Br, or I) in an aqueous phase to be converted into XMF, and then distributing the resulting hydrophobic XMF from the aqueous phase into an organic extraction solvent.

The non-patent document (ChemSusChem, 2015, 8, 1179-1188) described above relates to a technology to convert sugar ingredients in biomass to obtain CMF and reobtain a derivative in the form of AMF from CMF, in which case AMF is prepared through a substitution reaction between CMF and alkyl ammonium acetate.

In the AMF preparation process in the non-patent document described above, TCE is used to separate and extract CMF from the two-phase solvent system, and CMF purified through the TCE removal reacts with alkyl ammonium acetate to ultimately prepare AMF.

Methods of preparing AMF, including those in the case involving the use of such biomass-derived CMF, typically require separation and purification to be performed as preprocesses for CMF raw materials before being converted into AMF. During this process, there have been problems with increased costs resulting from the construction of separation and purification equipment and increased process steps. In addition, TCE and the like, polar organic solvents currently available as extraction solvents for CMF raw materials, are regulated when it comes to industrial use because there is a limit concentration set to 10 ppm or lower due to the carcinogenicity thereof.

Hence, the applicants have confirmed that high yields can be applied by directly applying the extraction solvent containing XMF, a raw material for AMF preparation, to the conversion reaction into AMF without performing separation and purification and that the yield improvement effect can be multiplied by selecting halogen species of XMF or adjusting XMF/AcO molar ratios, thus obtaining process convenience and economic feasibility, which had led to the present disclosure.

Hereinafter, a method of preparing AMF of the present disclosure will be described in detail.

The method of preparing AMF from a sugar ingredient of the present disclosure is characterized by including the following steps: (a) in a two-phase solvent system including an aqueous phase and an organic phase of an organic extraction solvent having a relative polarity of 0.2 or less based on a polarity of water at 1, converting a sugar ingredient to prepare XMF from the aqueous phase and then extracting the prepared XMF into the organic phase; and (b) adding acetate ions (AcO⁻) to the organic phase extracted directly in Step (a) to convert the extracted XMF into AMF.

Step (a) is to convert the sugar ingredient to prepare XMF from the aqueous phase in the two-phase solvent system including the aqueous phase and the organic phase of the organic extraction solvent and then to extract the prepared XMF into the organic phase.

This XMF, a reactant in a substitution reaction converted into AMF, is produced by bonding halogen ions and HMF resulting from a dehydration reaction of the sugar ingredient in an aqueous environment, and the resulting XMF is immediately extracted into the organic phase, thereby preventing the reaction from further proceeding to increase XMF yield.

In this case, the organic extraction solvent having a relative polarity of 0.2 or less compared to water is used, so the organic extraction solvent is not mixed with water and exists as a separate organic phase while facilitating the extraction of the resulting XMF in the aqueous phase into the organic phase.

In addition, the technical feature of the present disclosure is that the extraction solvent is immediately applicable to the conversion into AMF without the need to be separated from the organic phase containing the extracted XMF. According to this, the separation and purification processes of the extraction solvent are not involved, thus simplifying the process and reducing costs, which is advantageous.

In existing processes, XMF is first extracted, an extraction solvent is then removed to separate and purify the extracted XMF, and the resulting XMF is reapplied to an AMP preparation process, unlike the present disclosure. However, one feature of the present disclosure is that after extracting XMF using the organic solvent, the reaction proceeds in the organic phase or in the crude product form of XMF without being separated and purified by separately removing the extraction solvent.

The sugar ingredient, a raw material ingredient that is convertible into HMF and XMF, may have a concentration in the range of 0.01 to 20 wt % and may, for example, include one or more from: monosaccharides including glucose, fructose, and galactose; disaccharides including maltose, sucrose, and lactose; and polysaccharides including cellulose, hemicellulose, and starch including hexoses. Preferably, the sugar ingredient is one or more from glucose and cellulose.

The reaction with HMF to produce XMF may proceed in the presence of a hydrogen halide (HX), an alkali metal or alkaline earth metal chloride mixed with a hydrogen halide, or an alkali metal or alkaline earth metal chloride mixed with sulfuric acid (H₂SO₄), but is not limited thereto.

The halogen species (X) of the hydrogen halide may be one or more selected from F, Cl, Br, and I and, preferably, is Br.

Depending on the halogen species of the introduced hydrogen halide, the XMF resulting from the reaction between the HX and HMF may be one or more from fluoromethylfurfural (FMF), CMF, BMF, and iodomethylfurfural (IMF) and, preferably, is BMF.

The organic extraction solvent is configured to extract HMF having been converted into the halogen-bonded XMF to prevent the HMF, obtained from biomass, from being converted into levulinic acid or the like. The organic extraction solvent used preferably has a relative polarity of 0.2 or less based on a polarity of water at 1.

The extraction solvent used may include one or more from toluene, xylene, chlorobenzene, cyclohexane, pentane, hexane, heptane, methyl t-butyl ether, diethylamine, dioxane, and N,N-dimethylaniline. Preferably, one or more from toluene, xylene, and chlorobenzene are used in terms of environmental toxicity and solvent recovery.

Step (b) is to add the acetate ions (AcO) to the organic phase extracted directly in Step (a) to convert the extracted XMF into AMF.

The acetate ions (AcO⁻) in Step (b) may be derived from acetate salts such as NaOAc. However, the acetate ions (AcO⁻) are preferably present as exchange groups in a renewable anion exchange resin because the solubility of acetate salts, that is, inorganic salts, is low in the extraction solvent having a relative polarity of 0.2 or less, resulting in poor reactivity. The acetate ions (AcO⁻) may be present in the resin without limitation as long as the substitution reaction with XMF is possible, but may, for example, be ionically bonded by pairing with cations in the exchange resin or present at least one place on the surface of the resin or inside the resin.

The conversion efficiency of XMF into AMF is directly related to the substitution between the halogen ions (X) present in XMF and the acetate anions (AcO⁻) in the exchange resin and thus may depend on factors such as halogen species, XMF/AcO molar ratios, and the like.

As described above, the halogen species present in the XMF may be one or more from F, Cl, Br, and I but, preferably, is Br in terms of improving AMF yield and conversion speed.

Furthermore, the molar ratio of XMF in the organic phase to the acetate anions (AcO), that is, the XMF/AcO⁻ molar ratio, may be in the range of 0.01 to 0.99, which is preferably in the range of 0.25 to 0.70 and more preferably in the range of 0.25 to 0.45, in terms of improving the AMF yield and conversion speed.

In the meantime, in one example of the present disclosure, the anion exchange resin, in which anions are exchanged with acetate ions (AcO⁻) in the aqueous phase, may be characterized by being substituted through solvent substitution. The moisture contained in the anion exchange resin causes side reactions in which XMF, the reactant, is hydrolyzed before the conversion reaction into AMF to produce the hydrogen halide (HX) and further facilitates the hydrolysis of XMF by HX serving as an acid catalyst, leading to decomposition into by-products, such as levulinic acid or formic acid, or condensation polymerization into the form of polymers (humins). Thus, when using the anion exchange resin substituted through solvent substitution, the AMF yield can be improved.

The solvent substitution is a step of bringing one or more solvents selected from a ketone, an alcohol, and an ether that are miscible solvents in water and the extraction solvent, into contact with the anion exchange resin to mix the water contained in the exchange resin in the solvent, thereby reducing the moisture content in the exchange resin. In detail, when the anion exchange resin containing water makes contact with the miscible solvent in water and the extraction solvent, the water contained in the exchange resin migrates to the solvent, decreasing the moisture content in the exchange resin. The anion exchange resin substituted through solvent substitution makes contact with the organic extraction solvent having a relative polarity of 0.2 or less, contained in the extracted XMF organic phase, and then used in the reaction to convert XMF into AMF.

The method of controlling moisture in the anion exchange resin using solvent substitution is advantageous in that there is no change in the physical structure of the ion exchange resin, such as pore closure, compared to methods using vacuum drying.

As the solvent for the solvent substitution, the ketone used may, for example, include one or more selected from acetone, methyl ethyl ketone, diethyl ketone, methyl propyl ketone, ethyl propyl ketone, methyl isopropyl ketone, and methyl isobutyl ketone, the alcohol used may, for example, include one or more selected from methanol, ethanol, propanol, isopropanol, butanol, and pentanol, and the ether used may, for example, include one or more selected from tetrahydrofuran and dioxane.

Additionally, the moisture content in the anion exchange resin substituted through the solvent substitution may be reduced to a level in the range of 1 wt % to 0 wt % based on 100 wt % of the total moisture content in the anion exchange resin before the solvent substitution, thereby controlling the moisture content in the exchange resin.

After the solvent substitution, a step of removing the solvent used in the solvent substitution may be performed by means known in the art. Additionally, the present disclosure may be performed by reusing the anion exchange resin that has been used one or more times in the reaction without performing the solvent substitution described above. In this case, water contained in the exchange resin is consumed as the conversion reaction into AMF proceeds. For this reason, such an anion exchange resin reused after one or more times of use may also result in a higher AMF yield.

The conversion of the XMF into AMF may be performed in a temperature range of room temperature to 110° C. and, preferably, is performed in the temperature range of room temperature to 70° C. When the reaction temperature falls within the range of room temperature to 110° C., AMF may be obtained in high yields while reducing energy consumption.

In the meantime, the present disclosure may provide a method of preparing FDCA from a sugar ingredient, which includes the technical features of the method of preparing AMF from the sugar ingredient described above.

The method of preparing FDCA from the sugar ingredient of the present disclosure is characterized by including the following steps: (A) in a two-phase solvent system including an aqueous phase and an organic phase of an organic extraction solvent having a relative polarity of 0.2 or less based on a polarity of water at 1, converting a sugar ingredient to prepare XMF from the aqueous phase and then extracting the prepared XMF into the organic phase; (B) adding acetate ions (AcO⁻) to the organic phase extracted directly in Step (A) to convert the extracted XMF into AMF; (C) recovering the resulting AMF converted from the organic phase; and (D) converting the recovered AMF into FDCA through oxidation.

Steps (A) and (B) are described in the method of preparing AMF from the sugar ingredient, so redundant descriptions thereof will be omitted herein.

Step (C) is to recover the resulting AMF from the organic phase made of the extraction solvent, wherein the AMF recovery method may be performed by existing separation means, involving vacuum distillation or heating distillation or the use of a separator to remove the organic extraction solvent.

Step (D) is to convert the recovered AMF into FDCA through oxidation, wherein the AMF may be converted into FDCA through an oxidation reaction.

An oxidation catalyst may be used for the oxidation reaction. Any oxidation catalysts commonly used in the art may be used without limitation, but a platinum-supported carbonaceous catalyst (Pt/C) preferably is used.

In addition, a weak base may be used for the oxidation reaction. The weak base can keep the pH to a level of 7 or higher from FDCA obtained through the oxidation reaction and acetic acid being by-products and can prevent side reactions. Furthermore, the solubility of FDCA increases under weakly basic conditions compared to acidic or neutral aqueous conditions, so the amount of FDCA deposited on the surface of the oxidation catalyst significantly decreases, thereby improving catalytic reactivity and enabling the recovery and reuse of the catalyst.

The weak base used may be sodium bicarbonate (NaHCO₃), potassium bicarbonate (KHCO₃), sodium carbonate (Na₂CO₃), potassium carbonate (K₂CO₃), calcium carbonate (CaCO₃), magnesium carbonate (MgCO₃), calcium hydroxide (Ca(OH)₂), magnesium hydroxide (Mg(OH)₂), and mixtures thereof. Preferably, sodium bicarbonate (NaHCO₃) is used.

In addition, an oxidizing agent may be additionally added to the oxidation reaction. For example, non-limiting examples of known oxidizing agents including potassium permanganate (KMnO₄), hydrogen peroxide, benzoyl peroxide, peracetic acid, performic acid, perbenzoic acid, sodium hypochlorite (NaOCl), manganese oxide (MnO₂), potassium persulfate (K₂S₂O₈), ammonium cerium (IV) nitrate, ammonium peroxydisulfate, tert-butyl hydroperoxide, tert-butyl hypochlorite, chromium trioxide (CrO₃), cumene hydroperoxide, osmium tetroxide (OsO₄), potassium monopersulfate (KHSO₅), and sodium percarbonate may be used.

The oxidation reaction may be performed under the following conditions: a partial pressure of oxygen in the range of 1 to 20 bar, preferably in the range of 1 to 15 bar, or a partial pressure of air in the range of 1 to 50 bar, preferably in the range of 1 to 40 bar, and a temperature in the range of 70° C. to 130° C., preferably in the range of 90° C. to 120° C.

Hereinafter, the methods of preparing AMF and FDCA from the sugar ingredient will be described through one example of the present disclosure. For reference, although the following examples are provided to illustrate one or more preferred embodiments of the present disclosure, the present disclosure is not limited thereto. Various modifications can be made to the following examples falling within the scope of the present disclosure.

### Example 1: AMF Preparation from Toluene Organic Phase Containing Sugar Ingredient-Derived CMF

### Example 1-1: Obtaining of Toluene Organic Phase Containing Sugar Ingredient-Derived CMF

Into a one-necked flask, 0.60 g of glucose (99%, Sigma) and 1.17 g of NaCl (extra pure, DC Chemical), equal to six times the number of moles of glucose added to 6 mL (7.80 g) of a 10 M sulfuric acid aqueous solution, were introduced, followed by adding 30 mL of toluene (99%, Samchun, relative polarity: 0.099). Then, a condenser where ethanol at 0° C. circulated was installed to enable reflux.

The temperature inside the flask was then heated to the boiling point of toluene at 111° C. while stirring the mixed two-phase solution in the flask at 1400 rpm, followed by performing a dehydration reaction and reflux under atmospheric pressure. After 300 minutes of reaction time elapsed, the reaction flask was placed in a cold water bath at 0° C. for rapid cooling, and the toluene layer was then separated from the water layer. Anhydrous magnesium sulfate was added to the separated toluene layer to remove moisture, and then the toluene layer was obtained through filtration with the use of a filter. The resulting toluene layer was used as a CMF raw material for conversion into AMF without additionally performing separation and purification.

### Example 1-2: Preparation of Acetate Ion (OAc−)-Containing Anion Exchange Resin

An acetate ion (OAc⁻)-containing anion exchange resin required in preparing AMF from CMF was prepared as follows.

To 200 ml of a 2 M sodium acetate (NaOAc⁻) aqueous solution, 10 ml of an anion exchange resin (AmberChrom® 1×8 chloride form, Sigma-Aldrich) was added. Then, the resulting solution was stirred at room temperature for 24 hours and filtered through vacuum filtration to recover an anion exchange resin involving substitution with acetate ions. The acetate ion-containing anion exchange resin recovered in such a manner made contact with excess acetone several times and then with toluene, serving as an extraction solvent used for CMF extraction, to obtain the final acetate ion-containing anion exchange resin.

### Example 1-3: Obtaining of AMF-Containing Reaction Product Solution Using Toluene Organic Phase Extracted Directly in Example 1-1

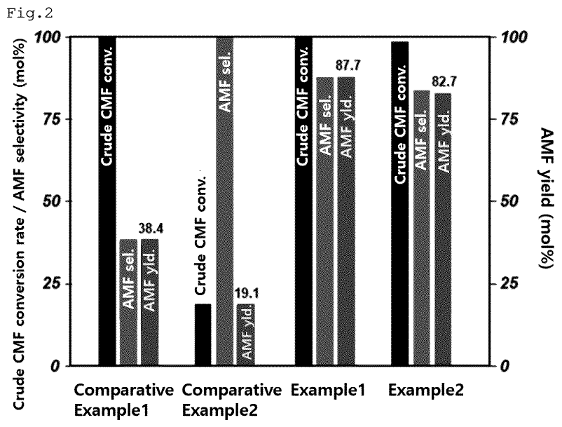

To convert CMF into AMF, 14 mL (1.2 meq AcO/mL) of the acetate ion-containing anion exchange resin obtained in Example 1-2 was added to 50 g of toluene containing CMF at a concentration of 1 wt %, obtained in Example 1-1, and then stirred at a reaction temperature of 90° C. for 6 hours at 1,000 rpm, thereby obtaining an AMF-containing reaction product solution (CMF/AcO molar ratio=0.30). CMF and AMF in the reaction product solution were quantified through analysis using a gas chromatograph (GC) equipped with a DB-624UI column and a flame ionization detector (FID) by employing the response factors measured between the analytes and 1-heptane as the internal standard substance. The selectivity and yield of the resulting AMF and the CMF conversion rate are shown in FIG. 2 and Table 1 below.

### Comparative Example 1

AMF was prepared through reactions in the same manner as in Example 1, except that the step of making contact with acetone in Example 1-2, the process of preparing the acetate ion-containing anion exchange resin, was not performed in Example 1.

### Example 2

AMF was prepared in the same manner as in Example 1, except that the acetate ion-containing anion exchange resin used once in Comparative Example 1 was reused.

### Comparative Example 2

AMF was prepared in the same manner as in Example 1, except that the acetate ion-containing anion exchange resin not involving solvent substitution was dried through vacuum drying (degree of vacuum <50 mmHg) in a dryer under conditions at 30° C. for 24 hours in Comparative Example 1.

FIG. 2 shows the CMF conversion rate, AMF selectivity, and AMF yield in Examples 1 and 2 and Comparative Examples 1 and 2 as one example of the present disclosure, as a graph.

Referring to FIG. 2, in Examples 1 and 2, in which the acetate ion-containing anion exchange resin made contact with acetone for solvent substitution, or the acetate ion-containing anion exchange resin is reused, the CMF conversion rate appeared to be 95% or higher, and the yield and selectivity of AMF appeared to be 80 mol % or higher.

On the other hand, in Comparative Example 1, the AMF yield significantly decreased to 38.4 mol %, and in Comparative Example 2, using the anion exchange resin dried through vacuum drying, the CMF conversion rate significantly decreased to less than 25%.

### Example 3: AMF Preparation 2 from Toluene Organic Phase Containing Sugar Ingredient-Derived CMF

### Example 3-1: Obtaining of Toluene Organic Phase Containing Sugar Ingredient-derived CMF

Into a three-necked flask, 10.0 g of glucose (99%, Sigma) and 19.48 g of NaCl (extra pure, DC Chemical), equal to six times the number of moles of glucose added to 100 mL (158.7 g) of a 10 M sulfuric acid aqueous solution, were introduced, followed by adding 200 mL of toluene (99%, Samchun, relative polarity: 0.099). Then, a condenser where ethanol at 0° C. circulated was installed to enable reflux.

The temperature inside the flask was then heated to the boiling point of toluene at 111° C. while stirring the mixed two-phase solution in the flask at 800 rpm, followed by performing a dehydration reaction and reflux under atmospheric pressure. After 300 minutes of reaction time elapsed, the reaction flask was placed in a cold water bath at 0° C. for rapid cooling, and the toluene layer was then separated from the water layer. Anhydrous magnesium sulfate was added to the separated toluene layer to remove moisture, and then the toluene layer was obtained through filtration with the use of a filter. The resulting toluene layer was used as a CMF raw material for conversion into AMF without additionally performing separation and purification.

### Example 3-2: Preparation of Acetate Ion (OAc−)-Containing Anion Exchange Resin

An acetate ion-containing anion exchange resin was obtained in the same manner as in Example 1-2.

### Example 3-3: Obtaining of AMF-Containing Reaction Product Solution Using Toluene Organic Phase Extracted Directly in Example 3-1

The acetate ion-containing anion exchange resin was added to the CMF-containing toluene solution obtained in Example 3-1 and then stirred at room temperature to perform a conversion reaction into AMF. As a result of the reaction, an AMF-containing reaction product solution (CMF/AcO molar ratio=0.30) was obtained.

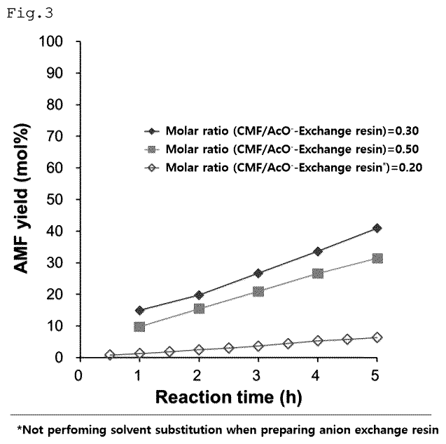

A sample was taken from the reaction product solution at a specific reaction time to measure the CMF conversion rate, AMF yield, and AMF selectivity. The results thereof are shown in FIG. 3 and Table 1 below.

### Example 4: AMF Preparation 3 from Toluene Organic Phase Containing Sugar Ingredient-Derived CMF

AMF was prepared in the same manner, except that the CMF/AcO molar ratio was adjusted to 0.50 in Example 3-3. The selectivity and yield of the resulting AMF and the CMF conversion rate from the reaction described above are shown in FIG. 3 below.

### Comparative Example 3: AMF Preparation 4 from Toluene Organic Phase Containing Sugar Ingredient-Derived CMF

A reaction product solution was prepared in the same manner, except that the acetate ion-containing anion exchange resin prepared without performing the step of making contact with acetone in Example 1-2, which is the process of preparing the acetate ion-containing anion exchange resin in Example 1, was adjusted to have a CMF/AcO molar ratio of 0.20. The selectivity and yield of the resulting AMF and the CMF conversion rate are shown in FIG. 3.

FIG. 3 shows the effects of the molar ratios of CMF/AcO in the exchange resin on the AMF yield and conversion speed depending on reaction time as one example of the present disclosure, as a graph.

Referring to FIG. 3, the higher the introduced amount of the acetate anion exchange resin in Examples 3 and 4, meaning that the CMF/AcO⁻ molar ratio decreased from 0.50 to 0.30, the conversion speed into AMF and the AMF yield appeared to increase.

In the meantime, in the case of Comparative Example 3, in which the molar ratio was 0.20, not only the conversion speed into AMF is insignificant as the reaction time elapses, but also the AMF yield fails to reach 10% after 5 hours of reaction time elapses, confirming that the removal of moisture through solvent substitution is a critical factor.

### Example 5: AMF Preparation 1 from Toluene Organic Phase Containing Sugar Ingredient-Derived BMF

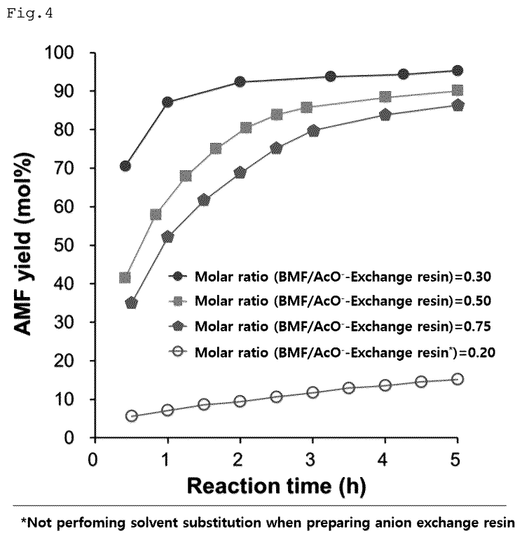

AMF was prepared in the same manner, except that 34.30 g of NaBr (extra pure, DC Chemical) instead of NaCl was introduced in Example 3-1 to prepare BMF, which was then used as the reaction raw material in Example 3-3. The selectivity and yield of the resulting AMF and the BMF conversion rate are shown in FIG. 4 and Table 2 below.

### Example 6: AMF Preparation 2 from Toluene Organic Phase Containing Sugar Ingredient-Derived BMF

AMF was prepared through reactions in the same manner, except that the BMF/AcO-molar ratio was adjusted to 0.50 in Example 5. The selectivity and yield of the resulting AMF and the BMF conversion rate are shown in FIG. 4.

### Example 7: AMF Preparation 3 from Toluene Organic Phase Containing Sugar Ingredient-Derived BMF

AMF was prepared through reactions in the same manner, except that the BMF/AcO-molar ratio was adjusted to 0.75 in Example 5. The selectivity and yield of the resulting AMF and the BMF conversion rate are shown in FIG. 4.

### Comparative Example 4: AMF Preparation 4 from Toluene Organic Phase Containing Sugar Ingredient-Derived BMF

AMF was prepared in the same manner, except that the acetate ion-containing anion exchange resin prepared without performing the step of making contact with acetone in the process of preparing the acetate ion-containing anion exchange resin was adjusted to have a BMF/AcO molar ratio of 0.20 in Example 5. The selectivity and yield of the resulting AMF and the BMF conversion rate are shown in FIG. 4.

FIG. 4 shows the effects of the molar ratios of BMF/AcO⁻ in the exchange resin on the AMF yield and conversion speed depending on reaction time as another example of the present disclosure, as a graph.

Referring to FIG. 4, it was confirmed that the higher the introduced amount of the acetate anion exchange resin in Examples 5 to 7, meaning that the BMF/AcO molar ratio decreased from 0.75 to 0.30, the conversion speed into AMF and the AMF yield appeared to increase. In the case of Example 5, in which the molar ratio was 0.30, the AMF yield exceeded 85% after 1 hour of reaction time elapsed, showing a significantly fast reaction speed.

On the other hand, in Comparative Example 4, in which the molar ratio was 0.20, it was confirmed that not only the conversion speed into AMF was insignificant as the reaction time elapsed, but also the AMF yield was only at a level of 15% after 5 hours of reaction time elapsed.

TABLE 1

| | | | | | h⁻¹ (number |
|---|---|---|---|---|---|
| | | | | | of moles |
| | | | | | of resulting |
| | | | BMF | AMF | AMF/(reaction |
| | | AMF | con- | selecti- | time * number |
| Classi- | Reaction | yield | version | vity | of moles |
| fication | time (h) | (%) | rate (%) | (%) | of AcO⁻)) |
| Example 3 | 1 | 14.9 | 17.5 | 85.1 | 0.044954 |
| (CMF/AcO⁻ | 2 | 19.8 | 22.8 | 86.7 | 0.029787 |
| exchange | 3 | 26.7 | 29.5 | 90.5 | 0.026786 |
| resin = 0.30) | 4 | 33.6 | 37.2 | 90.5 | 0.025324 |
| | 5 | 41.0 | 44.9 | 91.2 | 0.024665 |
| | 6 | 46.8 | 51.1 | 91.6 | 0.023493 |
| | 7 | 50.7 | 55.3 | 91.7 | 0.021815 |

TABLE 2

| | | | | | h⁻¹ (number |
|---|---|---|---|---|---|
| | | | | | of moles |
| | | | | | of resulting |
| | | | BMF | AMF | AMF/(reaction |
| | | AMF | con- | selecti- | time * number |
| Classi- | Reaction | yield | version | vity | of moles |
| fication | time (h) | (%) | rate (%) | (%) | of AcO⁻)) |
| Example 5 | 0.42 | 70.5 | 79.0 | 89.3 | 0.474344 |
| (BMF/AcO⁻ | 1 | 87.2 | 96.3 | 90.6 | 0.244231 |
| exchange | 2 | 92.4 | 99.5 | 92.8 | 0.129395 |
| resin = 0.30) | 3.25 | 93.8 | 99.9 | 93.8 | 0.080838 |
| | 4.25 | 94.3 | 100.0 | 94.4 | 0.062192 |
| | 5 | 95.3 | 100.0 | 95.4 | 0.05343 |

In addition, Tables 1 and 2 show the results of the conversion reaction into AMF in Examples 3 and 5 in which the XMF/AcO⁻ molar ratio is the same at 0.30, depending on the reaction time.

Referring to Tables 1 and 2, for the same reaction time of 1 hour, the number of moles of the resulting AMF per the number of moles of unit AcO per unit time was 0.244 h⁻¹ in Example 5 involving BMF as the reactant but was 0.045 h⁻¹ in Example 3 involving CMF, confirming that the conversion into AMF was significantly faster in Example 5 than in Example 3 by five or more times.

### Experimental Example 1: FDCA Preparation at Varying Reaction Temperatures

The AMF-containing reaction product solution obtained in Example 1 was filtered through vacuum filtration and separated from the anion exchange resin, and the toluene in the reaction product solution was subjected to vacuum distillation (room temperature, <50 mmHg) to recover AMF in a crude or crude product form. After adding 0.5 g of the crude AMF, 49.5 g of distilled water, 4 g of NaHCO₃ (99.7%, Sigma-Aldrich), and 0.5 g of Pt/C (5 wt % of Pt content, Sigma-Aldrich), an oxidation reaction was performed under the following conditions: a partial pressure of oxygen at 10 bar and a temperature in the range of 70° C. to 100° C. As a result, a reaction product solution containing FDCA and FFCA was obtained.

AMF, FDCA, and FFCA in the reaction product solution were analyzed using a high-performance liquid chromatograph (HPLC) equipped with an Aminex HPX-87H column and a refractive index (RI) and ultraviolet (UV) detector under the following conditions: an oven temperature of 60° C. and a mobile phase (5 mM sulfuric acid aqueous solution) flow rate of 0.6 ml/min. The yields of the resulting FFCA and FDCA and the AMF conversion rate in the reaction product solution, obtained through HPLC analysis, are shown in FIG. 3 and Table 3 below.

TABLE 3

| | Oxidation | | | |
|---|---|---|---|---|
| | reaction | AMF | FDCA | FFCA |
| | temperature | conversion | yield | yield |
| Reactant | (° C.) | rate (%) | (mol %) | (mol %) |
| AMF-containing | 70 | 74.2 | 34.4 | 28.2 |
| reaction product | 80 | 83.4 | 57.8 | 17.9 |
| solution in Example 1 | 90 | 95.5 | 81.3 | 6.1 |
| | 100 | 99.7 | 86.2 | 1.4 |

Table 3 shows that the higher the oxidation reaction temperature of AMF, the lower the FFCA yield and the higher the FDCA yield. The FDCA yield at a reaction temperature of 90° C. was improved by about 40% of the FDCA yield at a reaction temperature of 80° C. when compared thereto and, in particular, was significantly improved by about 130% of the FDCA yield at a reaction temperature of 70° C. when compared thereto.

Although the present disclosure has been described above with reference to the embodiments and the accompanying drawings, these embodiments are disclosed for illustrative purposes. Those skilled in the art will appreciate that various modifications, additions, and substitutions are possible, without departing from the scope and spirit of the invention as disclosed in the accompanying claims. Therefore, the scope of protection of the present disclosure should be defined by the appended claims.

1. A method of preparing 5-acetoxymethyl furfural (AMF) from a sugar ingredient, the method comprising:
(a) in a two-phase solvent system comprising an aqueous phase and an organic phase of an organic extraction solvent having a relative polarity of 0.2 or less based on a polarity of water at 1, converting a sugar ingredient to prepare 5-halomethyl furfural (XMF) from the aqueous phase and then extracting the prepared XMF into the organic phase; and
(b) adding acetate ions (AcO−) to the organic phase extracted directly in the (a) to convert the extracted XMF into AMF.

2. The method of claim 1, wherein the extraction solvent having a relative polarity of 0.2 or less in the (a) comprises one or more from toluene, xylene, and chlorobenzene.

3. The method of claim 1, wherein the XMF is bromomethylfurfural (BMF).

4. The method of claim 1, wherein the acetate ions (AcO−) in the (b) are present as exchange groups in an anion exchange resin.

5. The method of claim 4, wherein an XMF/AcO− molar ratio in the (b) is in a range of 0.01 to 0.99.

6. The method of claim 4, wherein the anion exchange resin is substituted through solvent substitution.

7. The method of claim 6, wherein the solvent substitution comprises substituting the anion exchange resin through solvent substitution using one or more solvents selected from a ketone, an alcohol, and an ether that are miscible solvents in water and the extraction solvent.

8. The method of claim 1, wherein the conversion of the extracted XMF into AMF is performed under a condition in a temperature range of room temperature (25° C.) to 110° C.

9. A method of preparing 2,5-furandicarboxylic acid (FDCA) from a sugar ingredient, the method comprising:
(A) in a two-phase solvent system comprising an aqueous phase and an organic phase of an organic extraction solvent having a relative polarity of 0.2 or less based on a polarity of water at 1, converting a sugar ingredient to prepare XMF from the aqueous phase and then extracting the prepared XMF into the organic phase;
(B) adding acetate ions (AcO−) to the organic phase extracted directly in the (A) to convert the extracted XMF into AMF;
(C) recovering the resulting AMF converted from the organic phase; and
(D) converting the recovered AMF into FDCA through oxidation.

10. The method of claim 9, wherein the oxidation in the (D) is performed under a condition in a temperature range of 70° C. to 130° C.

11. The method of claim 9, wherein the oxidation in the (D) is performed in the presence of a catalyst under a condition at 1 to 20 bar of oxygen or at 1 to 50 bar of air.
